SEQUENCE LISTING (12 sequences)

SEQ ID NO: 1 - DNA, 20 bp, Artificial Sequence
  forward primers of lipoprotein lipase
ccgccgaccaaagaagagat

SEQ ID NO: 2 - DNA, 20 bp, Artificial Sequence
  reverse primers of lipoprotein lipase
tagccacggactctgctact

SEQ ID NO: 3 - DNA, 20 bp, Artificial Sequence
  forward primers of PPARr2
atgggrgaaactctgggaga

SEQ ID NO: 4 - DNA, 25 bp, Artificial Sequence
  reverse primers of PPARr2
ctgtgtcaaccatggtcatttcttg

SEQ ID NO: 5 - DNA, 20 bp, Artificial Sequence
  forward primers of alkaline phosphatase
ccacgtcttcacatttggtg

SEQ ID NO: 6 - DNA, 20 bp, Artificial Sequence
  reverse primers of alkaline phosphatase
cagactgcgcctggtagttg

SEQ ID NO: 7 - DNA, 20 bp, Artificial Sequence
  forward primers of osteocalcin
ggcagcgaggtagtgaagag

SEQ ID NO: 8 - DNA, 20 bp, Artificial Sequence
  reverse primers of osteocalcin
agcagagcgacaccctagac

SEQ ID NO: 9 - DNA, 20 bp, Artificial Sequence
  forward primers of collagen type I
tttctagacggcaggtcagg

SEQ ID NO: 10 - DNA, 20 bp, Artificial Sequence
  reverse primers of collagen type I
aatccatcggtcatgctctc

SEQ ID NO: 11 - DNA, 20 bp, Artificial Sequence
  forward primers of GAPDH
ggctctccagaacatcatcc

SEQ ID NO: 12 - DNA, 20 bp, Artificial Sequence
  reverse primers of GAPDH
tttctagacggcaggtcagg

(19) United States
(12) Patent Application Publication
(10) Pub. No.: US 2017/0335288 A1
(43) Pub. Date: Nov. 23, 2017

(54) METHOD OF CLASSIFYING MESENCHYMAL STEM CELLS BY CONTROLLING CELL ADHESION, AND PROTEIN-COATED CULTURE CONTAINER THEREFOR

(71) Applicant: KOREA INSTITUTE OF SCIENCE AND TECHNOLOGY, Seoul (KR)

(72) Inventors: Sang-Heon KIM, Seoul (KR); JungMi KANG, Seoul (KR)

(21) Appl. No.: 15/602,083

(22) Filed: May 22, 2017

(30) Foreign Application Priority Data
May 23, 2016 (KR) 10-2016-0062811

(51) Int. Cl. C12N 5/0775; C08J 5/18; C12N 5/00; C12M 1/26

(52) U.S. Cl. CPC C12N 5/0662; C12N 5/0665; C12N 5/0068; C12N 5/0663; C12N 5/0668; C12N 5/0666; C08J 5/18; C12M 33/00; C12N 2500/32; C12N 2533/90

(57) ABSTRACT

Provided are a protein-coated culture container for classifying, identifying, or specifying mesenchymal stem cells by controlling cell adhesion; and a method of classifying, identifying, or specifying mesenchymal stem cells by using the container.

### CROSS-REFERENCE TO RELATED APPLICATION

This application claims the benefit of Korean Patent Application No. 10-2016-0062811, filed on May 23, 2016, in the Korean Intellectual Property Office, the disclosure of which is incorporated herein in its entirety by reference.

### BACKGROUND

### 1. Field

One or more embodiments relate to a protein-coated culture container for classifying, identifying, or specifying mesenchymal stem cells by controlling cell adhesion; and a method of classifying, identifying, or specifying mesenchymal stem cells by using the container.

### 2. Description of the Related Art

Adult stem cells are a small group of cells derived from various tissues, for example, brain, heart, lung, kidney, and spleen tissues, and differentiate into specific cell lines. Mesenchymal stem cells (MSCs), also referred to as multipotent mesenchymal cells or precursor cells, exist in most organs and tissues, e.g., bone marrow, fat, blood, muscle, and other connective tissues. Histologically, mesenchymal stem cells were the first group of cells to be isolated from stromal fractions of most connective tissues. A similar phenomenon has been observed in the differentiation of mesenchymal stem cells into various mesenchymal lineage cells, such as osteoblasts, adipocytes, and chondrocytes, as a reaction to culture conditions of the mesenchymal stem cells.

Mesenchymal stem cells have been reported to be successfully recognized by isolation of various cell groups having mesodermal multipotency. Marrow-derived mesenchymal stem cells are typically characterized by plastic-adhesive spindle-shaped cells of a single layer and are characterized by expression of CD29, CD90, CD105, and CD166 surface markers and low expression of CD34 and C45. Fat-derived matrix cells are found in stromal vascular fractions of subcutaneous fatty tissues and exhibit surface marker expression similar to that of the marrow-derived mesenchymal stem cells. Most studies for specifying particular stem cells have been performed by using a surface antigen-specific antibody. Some references have reported that cell differentiation potential varies depending on cell type and culture conditions of mesenchymal stem cells. However, no study has been conducted on the effects of an artificial matrix in controlling cell adhesion with respect to cell types and differentiation patterns, in order to specify the mesenchymal stem cells.

In this regard, the present inventors completed an embodiment of the present inventive concept by discovering that characteristics of mesenchymal stem cells may be classified, identified, or specified in a culture container by controlling cell adhesion by coating an artificial matrix for controlling cell adhesion on a culture container.

### SUMMARY

One or more embodiments include a method of classifying mesenchymal stem cells by controlling cell adhesion, wherein the method includes culturing mesenchymal stem cells in a culture container coated with a protein for controlling cell adhesion; analyzing characteristics of the mesenchymal stem cells thus cultured; and classifying the mesenchymal stem cells according to results of the analysis.

One or more embodiments include a culture container for classifying mesenchymal stem cells coated with a protein for controlling cell adhesion, wherein the protein for controlling cell adhesion is a protein for limiting integrin-mediated cell adhesion or for integrin-mediated cell adhesion.

Additional aspects will be set forth in part in the description which follows and, in part, will be apparent from the description, or may be learned by practice of the presented embodiments.

According to one or more embodiments, a method of classifying mesenchymal stem cells by controlling cell adhesion includes culturing mesenchymal stem cells in a culture container coated with a protein for controlling cell adhesion; analyzing characteristics of the mesenchymal stem cells thus cultured; and classifying the mesenchymal stem cells according to results of the analysis.

As used herein, the term “classification of mesenchymal stem cell” may denote classification or identification of cells according to characteristics of cells or specification of characteristics of cells. For example, conventionally, mesenchymal stem cells have been classified as CD29⁺ and CD34⁻ according to expression of a surface antigen marker of the cells, e.g., CD29, CD34, CD45, CD90, CD105, or CD166. The classification of mesenchymal stem cells, according to an embodiment, may include classification of mesenchymal stem cells by morphology of specific cells, intracellular signal transduction, or cell differentiation potential that occurs according to the control of cell adhesion.

The protein for controlling cell adhesion may be a protein for limiting integrin-mediated cell adhesion or for integrin-mediated cell adhesion. The integrin-mediated cell adhesion may denote that cells are cultured by being adhered to a culture container substantially through integrins existing on surfaces of the cells. The limiting of integrin-mediated cell adhesion may denote that cells are cultured by being adhered to a culture container substantially not through integrins existing on surfaces of the cells. The expression “substantially through integrins existing on surfaces of the cells” or “substantially not through integrins existing on surfaces of the cells” denotes that most of cells are adhered to a culture container with or without integrins as a medium or, and does not denote limiting the cells from being adhered to a culture container through another protein of the cells.

The culture container may be a culture container having a hydrophobic property on a surface thereof. The culture container having a hydrophobic property on a surface thereof may be a general cell culture container that is surface-treated with a polymer imparting a hydrophobic property to the container or a cell culture container formed of the polymer. The culture container may be a culture container coated with or having fixed thereon a protein for controlling cell adhesion. A detailed description of the culture container shall be provided later.

In one embodiment, the protein for limiting integrin-mediated cell adhesion may be a protein in which a polypeptide linker and a growth factor are fused. The limiting of integrin-mediated cell adhesion may denote adhesion with a fibroblast growth factor (FGF) receptor, a heparin sulfate proteolycan (HSPG), or a growth factor receptor, other than integrins, coated on a culture container, as a medium. For example, the limiting of integrin-mediated cell adhesion may denote that cells are adhered to a culture container through HSPG existing on surfaces of the cells. Thus, the culture container may have a protein, in which a polypeptide linker and a growth factor are fused, fixed to a surface of the culture container.

The polypeptide linker may be maltose-binding protein (MBP), hydrophobin, or hydrophobic cell-penetrating peptide (CPP). Also, the growth factor may be a vascular endothelial growth factor (VEGF), a fibroblast growth factor (FGF), an epidermal growth factor (EGF), a platelet-derived endothelial growth factor (PDGF), a hepatocyte growth factor (HGF), an insulin-like growth factor (IGF), or a heparin binding domain (HBD).

The protein for integrin-mediated cell adhesion may be an extracellular matrix protein. The extracellular matrix protein may be collagen, fibronectin, or laminin.

Fixation of a growth factor to a surface of a culture container may be achieved by any method known in the art that is used in fixing a polypeptide to a solid substrate surface and, traditionally, may involve physical adsorption or a covalent bond formed by a non-selective chemical reaction. Examples of the fixation method may include a method of fixing a protein by using a biotin-streptavidin/avidin bond by applying the protein to a solid surface treated with streptavidin or avidin after binding a biotin to the protein; a method of fixing a protein by collecting an active group (a chemical functional group for fixing a protein by a chemical bond) on a substrate using plasma; a method of fixing a protein by physical adsorption on a porous sol-gel thin film after forming the porous sol-gel thin film with a sufficiently increased surface area on a surface of the solid substrate by using a sol-gel method; a method of fixing an antithrombotic protein on a polytetrafluoroethylene (PTFE) surface by using a plasma reaction; a method of fixing a protein by binding at least two enzymes that are continuously fused to an enzyme having two cationic amino residues; a method of fixing a protein on a hydrophobic polymer layer that is bonded to a solid-phase support by using a substrate; a method of fixing a protein by using a buffer component on a plastic surface; and a method of fixing a protein by contacting the protein on a solid surface having a hydrophobic property in an alcohol solution.

In one embodiment, fixation may be performed in the form of a polypeptide linker-growth factor recombinant protein, in which an amino end of the growth factor is fused to a carboxyl end of the polypeptide linker, by using a polypeptide linker that may be recombinantly overexpressed and easily purified.

The extracellular matrix protein or the polypeptide linker-growth factor fused protein may be prepared by using chemical synthesis or a gene recombinant technique generally used in the art, or may be obtained by collecting a recombinant protein from a culture solution after culturing transformed bacteria that expresses the protein under appropriate conditions.

A process of fixing the protein thus obtained to a culture container does not require a particular treatment and may be actively performed by physical adsorption with a hydrophobic surface of the culture container using a hydrophobic domain located at an amino end of the polypeptide linker in a recombinant protein. The protein for controlling cell adhesion may be fixed to a surface of the culture container at a concentration in a range of 5 μg/ml to 100 μg/ml.

The culturing may include culturing by adhering mesenchymal stem cells to a culture container.

Before inoculating the mesenchymal stem cells on the culture container, cells proliferated by passaging may be used as the mesenchymal stem cells. A method of proliferating cells by passaging may be proliferating by passaging mesenchymal stem cells isolated by a common method by using a method general in the art. For example, the isolated mesenchymal stem cells may be cells that have been cultured in 1 to 10 passages, or cells that have been cultured in 10 or more passages.

In one embodiment, the mesenchymal stem cells may be isolated from various tissues or humans of various racesor ages. For example, the mesenchymal stem cells may be mesenchymal stem cells derived from adipose tissue, a placenta, umbilical cord blood, muscle tissue, corneal tissue, or bone marrow tissue. Also, for example, the mesenchymal stem cells may be adipose stem cells, bone marrow stem cells, neural stem cells, placental stem cells, or cord blood stem cells.

A concentration of inoculating the mesenchymal stem cells on a culture container may be in a range of 1.0×10³ cells/cm² to 1.0×10⁶ cells/cm². Also, a period of culturing may be in a range of 30 minutes to 30 days. The culturing period may be appropriately determined by those of ordinary skill in the art according to characteristics of cells to be analyzed, and, for example, may be cultured for about 14 days to 30 days to confirm differentiation potential. A medium suitable for the culturing may be any medium that is generally used in culturing and/or differentiating mesenchymal stem cells, as long as the medium is serum-containing or serum-free. For example, the medium may be prepared by adding a serum to a Dulbeco's modified eagle medium (DMEM), Ham's F12, or a mixture thereof. Also, the culturing may be performed in a culture medium for analyzing characteristics of the desired cells. For example, in order to analyze the specific cell differentiation potential of mesenchymal stem cells, for example, adiposite differentiation potential or osteoclast differentiation potential, the mesenchymal stem cells can be differentiated into specific cells, such as adipocytes or bone cells, by culturing the stem cells in a specific cell differentiation medium such as an adipocyte differentiation medium or an osteocyte differentiation medium.

The analyzing of characteristics of the mesenchymal stem cells may be analyzing cytoskeletal structures, cell morphology, intracellular signal transduction, or cell differentiation potential of mesenchymal stem cells.

Those of ordinary skill in the art may perform an appropriate method of analyzing characteristics of the desired cells. For example, differentiation potential of cells according to cell adhesion control may be analyzed by culturing and differentiating mesenchymal stem cells in a medium for inducing cell differentiation, and then staining the differentiated cells or analyzing whether a differentiation marker gene is expressed. Also, for example, when phosphorylation of a specific protein is analyzed, intracellular signal transduction according to cell adhesion control may be analyzed, and a cytoskeletal structure and cell morphology according to cell adhesion control may be analyzed using a microscope.

The classifying of the mesenchymal stem cells may include classifying the mesenchymal stem cells into mesenchymal stem cells that have particular characteristics according to cell adhesion control. For example, the classifying process may include a step that includes classifying mesenchymal stem cells having particular morphology or differentiation potential as cultured by integrin-mediated cell adhesion; or mesenchymal stem cells having particular morphology or differentiation potential as cultured with limited integrin-mediated cell adhesion

In one embodiment, since characteristics of mesenchymal stem cells may vary depending on control of characteristics of adhesion to a culture container of mesenchymal stem cells cultured on a culture container, the characteristics of the mesenchymal stem cells may be classified according to control of cell adhesion by using a method of classifying mesenchymal stem cells. For example, the mesenchymal stem cells may be classified or characterized in adipose stem cells or bone marrow stem cells that have a high phosphorylation ratio of FAK and are cultured through integrin-mediated adhesion or adipose stem cells that have adipocyte differentiation potential and are cultured with limited integrin-mediated adhesion.

According to one or more embodiments, provided is a culture container for classifying mesenchymal stem cells coated with a protein for controlling cell adhesion, wherein the protein for controlling cell adhesion is a protein for limiting integrin-mediated cell adhesion or for integrin-mediated cell adhesion.

The culture container is the same as described above. For example, a protein for limiting integrin-mediated cell adhesion is a protein in which a polypeptide linker and a growth factor are fused, or the protein for integrin-mediated cell adhesion may be an extracellular matrix protein.

In one embodiment, since mesenchymal stem cells have particular characteristics according to control of cell adhesion to a culture container during culturing of the mesenchymal stem cells, the culture container may be effectively used in classifying, identifying the mesenchymal stem cells, or specifying the mesenchymal stem cells.

### BRIEF DESCRIPTION OF THE DRAWINGS

These and/or other aspects will become apparent and more readily appreciated from the following description of the embodiments, taken in conjunction with the accompanying drawings in which:

FIG. 1 is a view that illustrates a method of classifying mesenchymal stem cells, according to an embodiment;

FIGS. 2A-2B are views that illustrate an adhesion percentage of adipose stem cells and bone marrow stem cells adhered to a culture container, according to an embodiment;

FIGS. 3A-3D are views that illustrate the results of analyzing intracellular signal transduction according to cell adhesion control, according to an embodiment;

FIG. 4 is a view that illustrates a cytoskeleton pattern according to cell adhesion control, according to an embodiment;

FIG. 5 is a view that illustrates the results of analyzing cell morphology according to cell adhesion control by using a scanning electron microscope (SEM), according to an embodiment;

FIGS. 6A-6F are views that illustrate the results of analyzing differentiation potential of stem cells according to cell adhesion control by using a cell dye, according to an embodiment; and

FIGS. 7A-7B are views that illustrate the results of measuring differentiation potential of stem cells according to cell adhesion control by using art-PCR, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

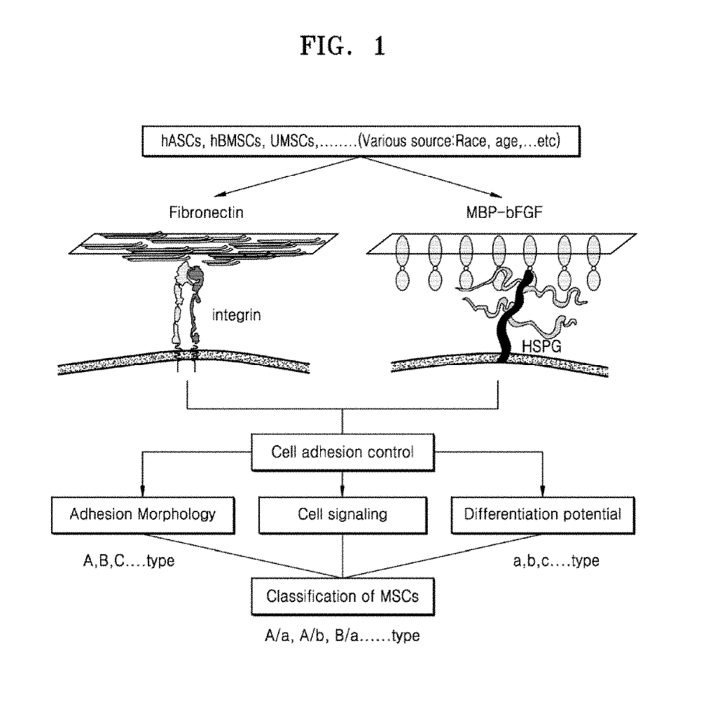

FIG. 1 is a view that illustrates a method of classifying mesenchymal stem cells, according to an embodiment. Referring to FIG. 1, the method of classifying mesenchymal stem cells may include culturing mesenchymal stem cells in a culture container coated with a protein for controlling cell adhesion; analyzing characteristics of the mesenchymal stem cells thus cultured; and classifying the mesenchymal stem cells according to the analyzed results.

In one embodiment, the mesenchymal stem cells may be isolated from various tissues or humans of various races or ages. For example, the mesenchymal stem cells may be mesenchymal stem cells derived from adipose tissue, a placenta, umbilical cord blood, muscle tissue, corneal tissue, or bone marrow tissue. Also, for example, the mesenchymal stem cells may be adipose stem cells, bone marrow stem cells, neural stem cells, placental stem cells, or cord blood stem cells.

In one embodiment, the protein for controlling cell adhesion may be a protein for limiting integrin-mediated cell adhesion or a protein for integrin-mediated cell adhesion. The integrin-mediated cell adhesion may denote that cells are cultured by being adhered to a culture container substantially through integrins existing on surfaces of the cells. The limiting of integrin-mediated cell adhesion may denote that cells are cultured by being adhered to a culture container substantially not through integrins existing on surfaces of the cells, for example, through HSPG existing on the surfaces of the cells.

In one embodiment, the protein for limiting integrin-mediated cell adhesion may be a protein in which a polypeptide linker and a growth factor are fused, for example, a MBP-FGF fused protein. Also, the protein for integrin-mediated cell adhesion may be an extracellular matrix protein, for example, fibronectin.

In one embodiment, the analyzing of characteristics of the mesenchymal stem cells may be analyzing cytoskeletal structures, cell morphology, intracellular signal transduction, or differentiation potential of the mesenchymal stem cells. Those of ordinary skill in the art may perform an appropriate method for analyzing characteristics of the desired cells. For example, differentiation potential of cells according to cell adhesion control may be analyzed by culturing and differentiating mesenchymal stem cells in a medium for inducing cell differentiation, and then staining the differentiated cells or analyzing whether a differentiation marker gene is expressed. Also, for example, when phosphorylation of a specific protein is analyzed, intracellular signal transduction according to cell adhesion control may be analyzed, and a cytoskeletal structure and cell morphology according to cell adhesion control may be analyzed using a microscope.

In one embodiment, the classifying of the mesenchymal stem cells may include classifying the mesenchymal stem cells into mesenchymal stem cells that have particular characteristics according to cell adhesion control. According to a method of classifying the mesenchymal stem cells, according to an embodiment, characteristics of the mesenchymal stem cells may be classified according to cell adhesion control. For example, the mesenchymal stem cells may be classified or characterized in adipose stem cells or bone marrow stem cells that have a high phosphorylation ratio of FAK and are cultured through integrin-mediated adhesion, or adipose stem cells that have adipocyte differentiation potential and are cultured while limiting integrin-mediated adhesion.

### Example 1. Culture of Stem Cells by Controlling Cell Adhesion Characteristics

(1.1) Preparation of Culture Container for Limiting Integrin-Mediated Adhesion and for Integrin-Mediated Adhesion

A recombinant protein fused with a maltose binding protein (MBP) as a linker for limiting integrin-mediated adhesion was coated on a surface of a culture container.

In particular, culture containers each having growth factors, such as a vascular endothelial growth factor (VEGF), a fibroblast growth factor (FGF), and a heparin binding domain (HBD) fixed on a surface of the culture containers by a linker MBP were prepared. The MBP and MBP-growth factor fused protein were prepared and purified according to descriptions disclosed in Han M et al., Design and characterization of a maltose binding protein-linked growth factor for matrix engineering, Biotechnology letters 2009; 31:1677-84, Kang et al., Adhesion and differentiation of adipose-derived stem cells on a substrate with immobilized fibroblast growth factor, Acta biomaterialia 2012; 8:1759-67., Kang et al., Control of mesenchymal stem cell phenotype and differentiation depending on cell adhesion mechanism, European cells & materials 2014; 28:387-403, and Park et al., The correlation between human adipose-derived stem cells differentiation and cell adhesion mechanism, Biomaterials 2009; 30:6835-43. These documents are incorporated herein in their entirety by reference. The purified recombinant MBP and MBP fused protein (MBP-VEGF, MBP-HBD, and MBP-bFGF) were filtered by using a 0.22 μm syringe filter (Millex GV, available from Millipore) on a clean bench (available from Sanyo). Then, 100 μl of the protein was added to a non-tissue culture treated 96-well plate (NTCP, which is formed of a polystyrene material and has a hydrophobic property on a surface, available from Falcon) at a concentration of 20 μg/ml and was left on the clean bench for 4 hours to fix the protein onto a surface of the plate.

In order to prepare a culture container for integrin-mediated adhesion, fibronectin (F1441, USA, available from Sigma) was dissolved in PBS at a concentration of 20 μg/ml, and 100 μl of the fibronectin was added to the 96-well plate to prepare the culture container as described above.

(1.2) Isolation and Culture of Stem Cells

Adipose stem cells (hASCs) and bone marrow stem cells (hBMSCs) were isolated as follows.

First, subcutaneous adipose tissues of a normal person supplied from the plastic surgery research laboratory of Catholic University were washed with PBS containing 1% penicillin/streptomycin (PS) three times and contaminated blood was removed. Thereafter, the blood-removed tissues were chopped using surgical scissors. These chopped tissues were added to a tissue lysing solution (DMEM/F-12, available from Welgene) containing 1% of BSA (w/v), 0.3% of collagenase type 1, and 1% of PS, and the solution was stirred (orbital shaking) at 37° C. for 1 hour. Then, the supernatant was discarded, and the cell suspension was filtered by using a 250 μm Nitex filter (available from Sefar America Inc.) to remove the tissue debris and was centrifuged at a rate of 1,000 rpm for 5 minutes. The cells collected from the centrifugation were cultured in a DMEM/F12 medium containing 10% FBS (available from Hyclone/Thermo Scientific, USA) and in a culture medium containing 100 U/ml of penicillin/streptomycin.

The hBMSCs (available from Celbio, Republic of Korea) was cultured in a high-glucose DMEM, a culture medium containing 10% FBS (available from Hyclone/Thermo Scientific, USA), and a culture medium containing 100 U/ml of penicillin/streptomycin.

In each passage, the plate included 5×10³ cells/cm² of cells, cultured until 70% of confluence, and subcultured by using 0.25% of trypsin-EDTA (available from Invitrogen). Five passages of hASCs and hBMSCs were used in every Example.

### Example 2. Analysis of Characteristics of Stem Cells According to Cell Adhesion Control Characteristics

(2.1) Analysis of Stemness

Before analyzing characteristics of stem cells according to cell adhesion control characteristics, expression types of adipose stem cells and bone marrow stem cells were analyzed by using a Beckman Coulter FACS (Cytomics FC 500, USA) to evaluate stemness.

In particular, the analysis was performed by using a laser line of a wavelength of 488 nm or 594 nm, and an apparatus used in the analysis was calibrated every day by using fluorescence latex particles. The hASCs and hBMSCs of five passages were washed with PBS containing 1% (w/v) of BSA. Thereafter, the resultant was stained by incubating at 4° C. for 60 minutes by using a conjugated primary human antibody or an appropriate IgG isotype with respect to CD29, CD14, CD45, CD31, CD34 (Beckman Coulter, USA), CD90 (BD biosciences, USA), CD105 (Caltac Laboratories, USA), and CD166 (BD biosciences, USA). After the staining, the cells were washed three times with PBS containing 1% of FBS, re-suspended with PBS again, and then analyzed by flow cytometry.

As a result, it was confirmed that the hASCs and hBMSCs expressed CD29 (91.2% of hASCs and 95.4% of hBMSCs), CD90 (97.7% of hASCs and 99.8% of hBMSCs), CD105 (99.9% of hASCs and 99.8% of hBMSCs), and CD166 (98.2% of hASCs and 100.0% of hBMSCs), which are markers of a typical mesenchymal substrate cell. Also, it was confirmed that hematopoiesis markers CD14 (0.2% of hASCs and 0.1% of hBMSCs) and CD45 (0.1% of hASCs and 0.2% of hBMSCs) and endothelial cell markers CD31 (0.0% of hASCs and 0.0% of hBMSCs) and CD34 (0.1% of hASCs and 0.0% of hBMSCs) were almost not expressed.

(2.2) Selection of Culture Container for Limiting Integrin-Mediated Adhesion

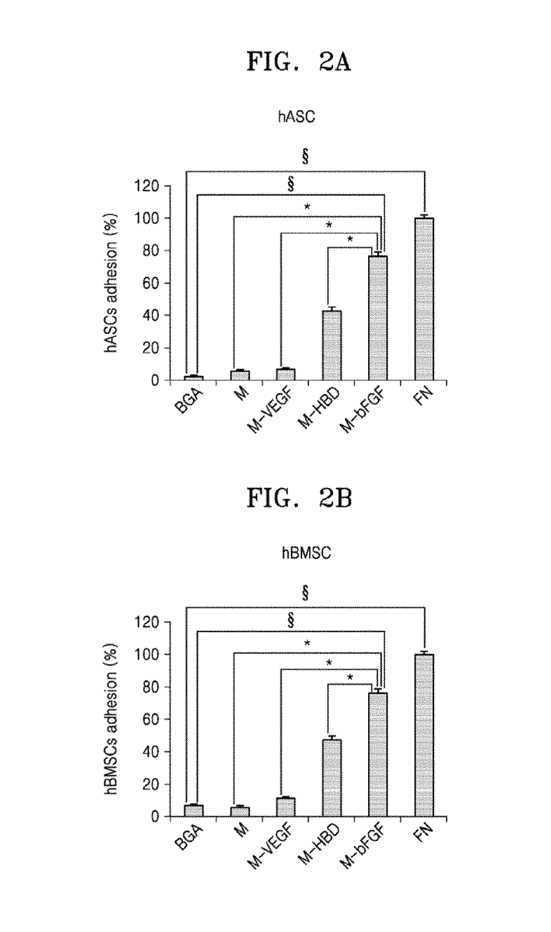

The hASCs and hBMSCs were inoculated in the culture containers thus prepared (FN, MBP, MBP-VEGF, MBP-HBD, MBP-bFGF) at a concentration of 2×10⁴ cells/cm², and the cells were allowed to adhere under conditions of 5% CO₂ and 37° C. for 1 hour to select a culture container for limiting integrin-mediated adhesion by using a heparin binding affinity. A BSA-coated well plate was used as a control group. Cells not adhered to the container were removed by washing each well with PBS, and a protein was extracted from the adhered cells by using a lysis solution (0.25% of NaOH, and 0.5% of SDS). A percentage of cells adhered to a surface of each well were indirectly determined by measuring a protein concentration by using a BCA assay kit (available from Pierce, Rockford, Ill.). An absorbance of each well was measured at a wavelength of 562 nm by using a UV-microplate reader (model VERSA max; Molecular Device). A percentage of the adhered cells was determined based on a percentage of 100% of cells adhered to FN, and the results are shown in FIG. 2.

FIGS. 2A-2B are views that illustrate an adhesion percentage of adipose stem cells and bone marrow stem cells adhered to a culture container, according to an embodiment.

As shown in FIGS. 2A-2B, the hASCs and hBMSCs in the culture container coated with M-bFGF had adhesion potentials of 78±2% (FIG. 2A) and 79±2% (FIG. 2B), respectively; the hASCs and hBMSCs in the culture container coated with M-VEGF had adhesion potentials of 8±2% (FIG. 2A) and 12±2% (FIG. 2B), respectively; and the hASCs and hBMSCs in the culture container coated with M-HBD had adhesion potentials of 42±3% (FIG. 2A) and 49±2% (FIG. 2B), respectively. Thus, in the Examples hereinafter, the culture container coated with M-bFGF, which showed the best adhesion potentials, was used as a culture container having limited integrin-mediated adhesion.

(2.3) Intracellular Signal Transduction Analysis According to Cell Adhesion Control

Western-blotting for phosphorylation detection of FAK and ERK1/2 was performed to evaluate intracellular signal transduction according to cell adhesion control.

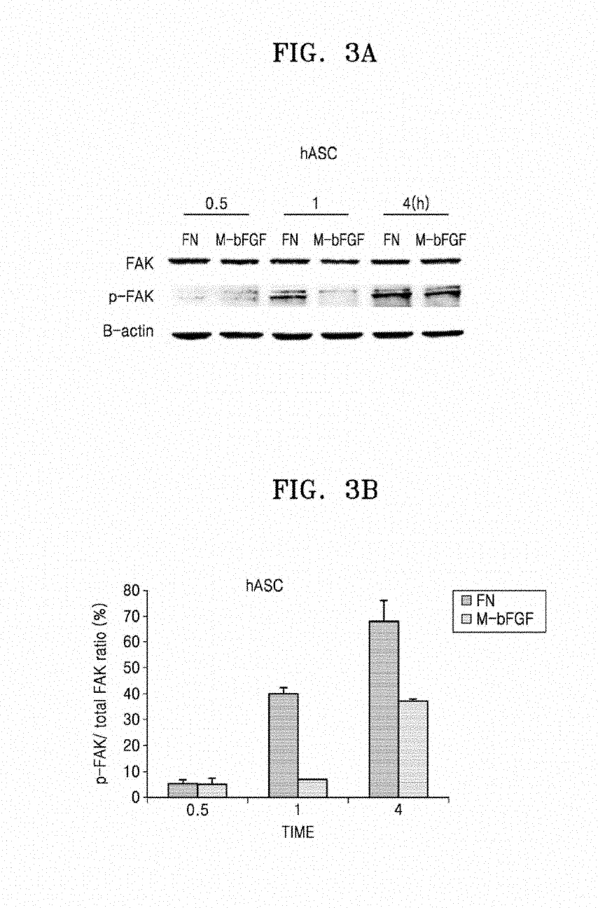

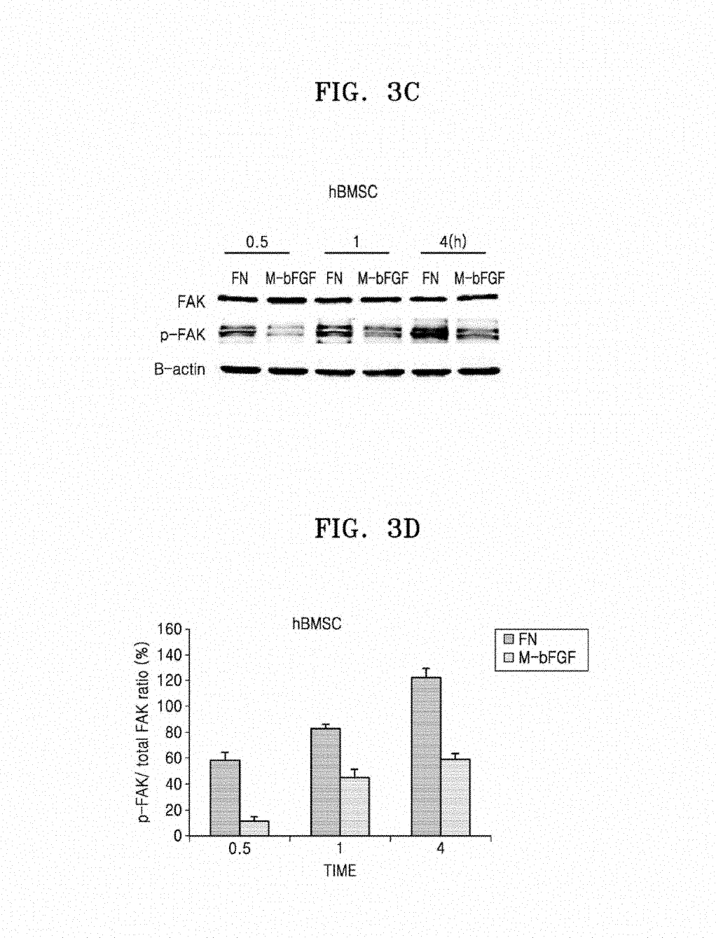

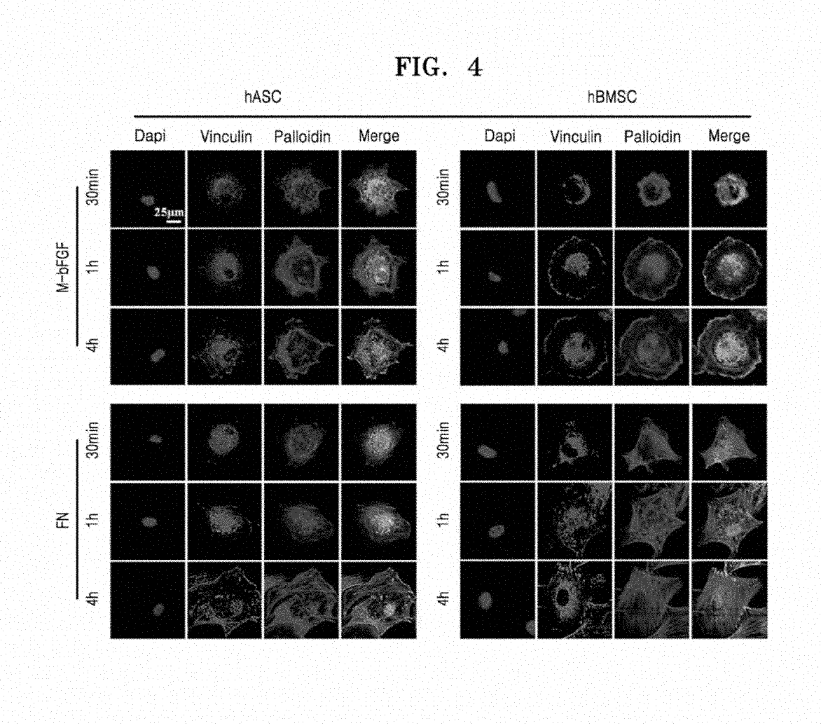

In particular, the hASCs and hBMSCs adhered to M-bFGF and FN under the conditions of 37° C. and 5% CO₂ for a predetermined time were removed from each well by using a cold RIPA buffer (R0278, Sigma Aldrich) containing protease inhibitor cocktail (P8340, Sigma Aldrich) and phosphatase inhibitor cocktail (P5726, Sigma Aldrich), and the cells were dissolved at 4° C. for 1 hour. A lysate was centrifuged at 4° C. for 30 minutes at 15,000×g, diluted in a Laemmli sample buffer (Bio-Rad, Hercules, USA), and heated at 95° C. for 5 minutes. Then, a protein was isolated by SDS-PAGE using an 8% resolving gel and placed in a nitrocellulose membrane (Bio-Rad, Hercules, USA). The membrane, together with a primary antibody, FAK, p-FAK, ERK1/2, p-ERK1/2, and β-actin (Cell signaling Technology), was incubated at 4° C. for overnight. For the detection, peroxydase-conjugated anti-mouse IgG or anti-rabbit IgG, and an ECL method (Pierce, USA), were used according to instructions of the manufacturer. The membrane was scanned by using LAS3000 (Fuji film, Japan) to produce a chemiluminescence image. For quantitative analysis of phosphorylated proteins (p-FAK and p-ERK1/2), the total amount of the proteins and a pixel density of the phosphorylated proteins were compared by using image J software (NIH, Bethesda, Md.), and the results are shown in FIG. 3A-3D.

FIGS. 3A-3D are views that illustrate the results of analyzing intracellular signal transduction according to cell adhesion control, according to an embodiment.

As shown in FIGS. 3A-3D, it may be known that, in the case of the hASCs, a p-FAK/total FAK ratio in an FN-coated culture container was higher than that in a MBP-bFGF-coated culture container (FIG. 3A and FIG. 36). Also, it may be known that, in the case of the hBMSCs, a p-FAK/total FAK ratio in an FN-coated culture container was higher than that in a MBP-bFGF-coated culture container (FIG. 3C and FIG. 3D). Also, it may be confirmed that an amount of the phosphorylated FAK detected by the hASCs adhered to FN was less than that of the hBMSCs adhered to FN. Also, it may be known that FAK phosphorylation is further limited in the stem cells adhered to FN than in the stem cells adhered to MBP-bFGF.

In this regard, it may be known that intracellular signal transduction may occur differently in the stem cells cultured by integrin-mediated cell adhesion and the stem cells cultured by limited integrin-mediated cell adhesion.

(2.4) Evaluation of Cytoskeleton of Cells According to Cell Adhesion Control

Cell surface integrins transduce various extracellular signals through FA molecules connected to an actin cytoskeletal structure. Thus, in order to evaluate a cytoskeleton of a cell according to cell adhesion control, a focal adhesion assay was performed.

In particular, the hASCs and hBMSCs were dispensed on M-bFGF-coated and FN-coated cover slips at a concentration of 1.0×10⁴ cells/cm² and were cultured under conditions of 37° C. and 5% CO₂ for a predetermined time. The cells were washed with PBS twice, fixed in 4% paraformaldehyde (P6148. Sigma Aldrich) for 8 to 10 minutes, submerged in 0.2% (v/v) Triton-X 100(T8787, Sigma Aldrich)/PBS for 10 minutes, and blocked by using 2% (v/v) BSA/PBS at room temperature for 1 hour. Then, the cells were washed again and incubated with 0.67 μg/ml of mouse monoclonal anti-vinculin (700062, Life Technologies Invitrogen) at 37° C. for 1 hour. Subsequently, the cells were washed with PBS three times and incubated with 10 μg/ml of fluorescein isothiocyanate (FITC)-conjugate goat anti-mouse IgG (chemicon International, Temecula, Calif.), and 37.5 ng/ml of tetramethyl rhodamine isothiocyanate (TRITC)-conjugate (phalloidin) (P1951, Sigma Aldrich) at room temperature for 1 hour. Next, the cells were washed with PBS three times, incubated for 5 minutes with a 4,6-diamidino-2-phenylindole (DAPI) solution, and then washed again several times with PBS. The fluorescence-dyed cells were analyzed by using a confocal microscope. Three independent samples were evaluated by each group, and the images were taken by using the confocal microscope. The results are shown in FIG. 4.

FIG. 4 is a view that illustrates a cytoskeletal pattern according to cell adhesion control, according to an embodiment.

As shown in FIG. 4, it may be confirmed that the FA molecules (vinculin) had improved distribution and formation in the whole cytoplasm in cells cultured from the FN-coated culture container than in the cells cultured from the MBP-bFGF-coated culture container. It may be confirmed that vinculin expression in the hASCs was shown to be well localized around nuclei at an initial stage and then, as the culture time increased, were distributed peripherally as bundles forming a polygonal shape. On the other hand, it may be confirmed that vinculin expression of the hBMSCs was shown to be distributed peripherally to form a circular line.

It may be known that a cytoskeletal structure and cell distribution may vary according to the cell adhesion control.

(2.5) Analysis of Cell Morphology According to Cell Adhesion Control

Filopodia and lamellipodia mediate initial cell adhesion and distribution, and thus determine cell morphology. Thus, a scanning electron microscope was used to analyze cell morphology according to cell adhesion control.

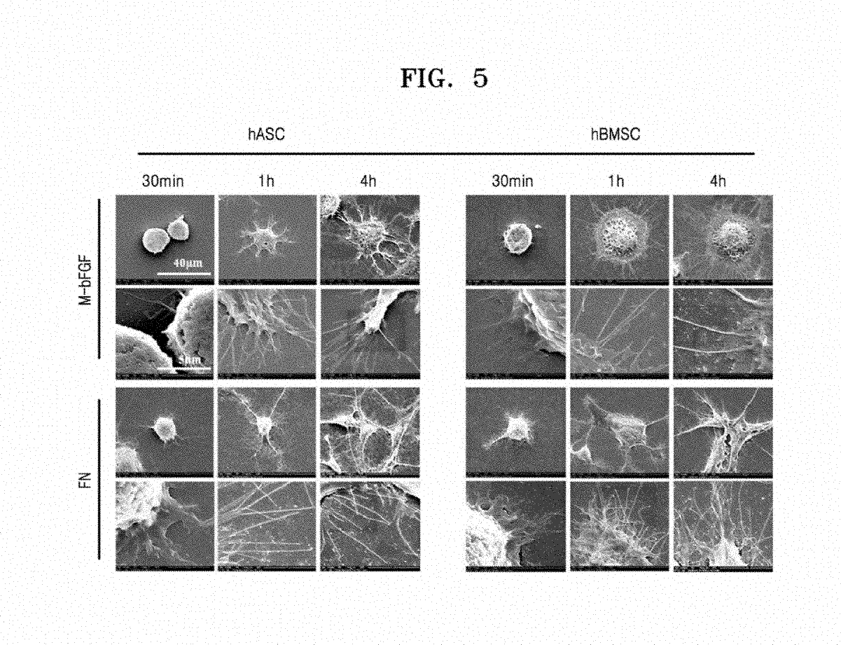

In particular, the hASCs and hBMSCs were dispensed on M-bFGF-coated and FN-coated cover slips at a concentration of 1.0×10⁴ cells/cm² and were cultured under conditions of 37° C. and 5% CO₂ for a predetermined time. The cells were gently washed with PBS three times at each time point (30 minutes, 1 hour, and 4 hours) and fixed in an SEM-level glutaaldehyde at 4° C. for 30 minutes. The cover slips were impregnated in 2% osmium tetroxide in ionized water for 30 minutes to perform a secondary fixing process. Then, the fixed cells were dehydrated twice by using alcohol of a series of different concentrations (30%, 50%, 70%, 80%, 90% and 100%). After the dehydration, the cells were placed in hexamethyldisilazane (HDMS) for 2 minutes and vacuum-dried for 1 night. In order to obtain an SEM image, the cells were sputter-coated with gold at 10 mA for 60 seconds, and the image was obtained by using an Inspect F50 (Zeus) at 15 kV. The results are shown in FIG. 5.

FIG. 5 is a view that illustrates the results of analyzing cell morphology according to cell adhesion control by using a scanning electron microscope (SEM), according to an embodiment.

As shown in FIG. 5, in the initial cell adhesion stage (30 minutes), it may be confirmed that the hASCs showed weak adhesion characteristics with respect to M-bFGF, together with a small number of filopodia adhered to a surface. At the final time point (4 hours), the cells had a spherical shape and filopodia of a length of about 45 to 55 μm located at poles of the cells with respect to M-bFGF. On the other hand, the hBMSCs had adhesion, distribution, and shape characteristics different from those of the hASCs. In particular, in the initial cell adhesion stage (30 minutes), unlike in the case of the hASCs, lamellipodia was observed in the case of the hBMSCs with respect to a surface of the M-bFGF. In the initial cell adhesion stage, it may be confirmed that the cells were adhered to a surface by the protruding lamellipodia. As time progressed, the cells were well distributed with lamellipodia of a ring shape, and filopodia of about 20 to 34 μm were observed at the front edge of the lamellipodia. It may be confirmed that, as time progressed, a shape of lamellipodia remained the same, while a length of filopodia increased by up to about 45 to 55 μm.

As a result, it may be known that cell membranes and cytoskeletal structures may vary according to cell adhesion control, which allows classification of stem cells.

(2.6) Analysis of Differentiation Potential According to Cell Adhesion Control

Adipose cell differentiation potential and bone marrow cell differentiation potential according to cell adhesion control were evaluated as follows.

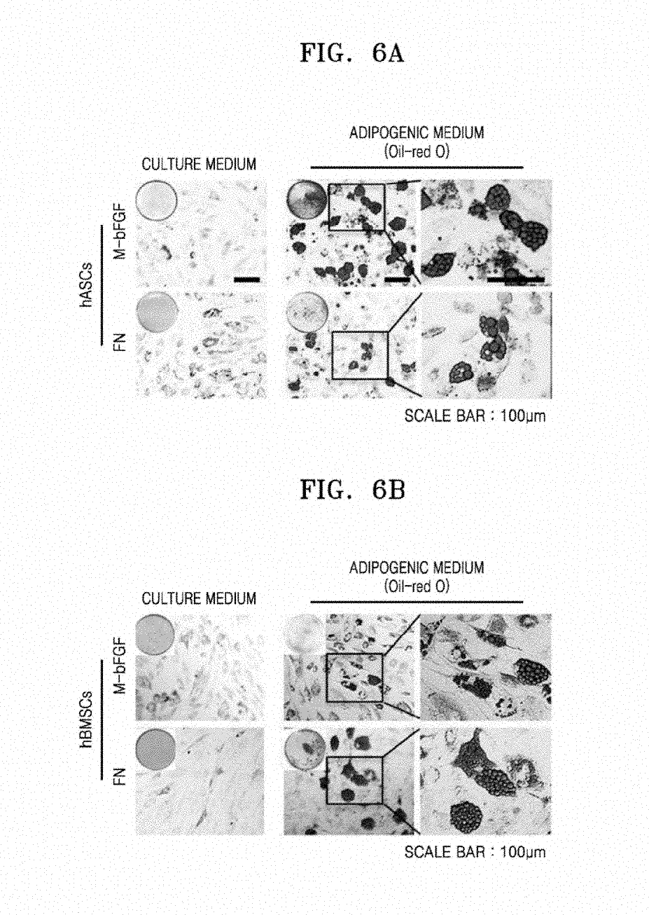

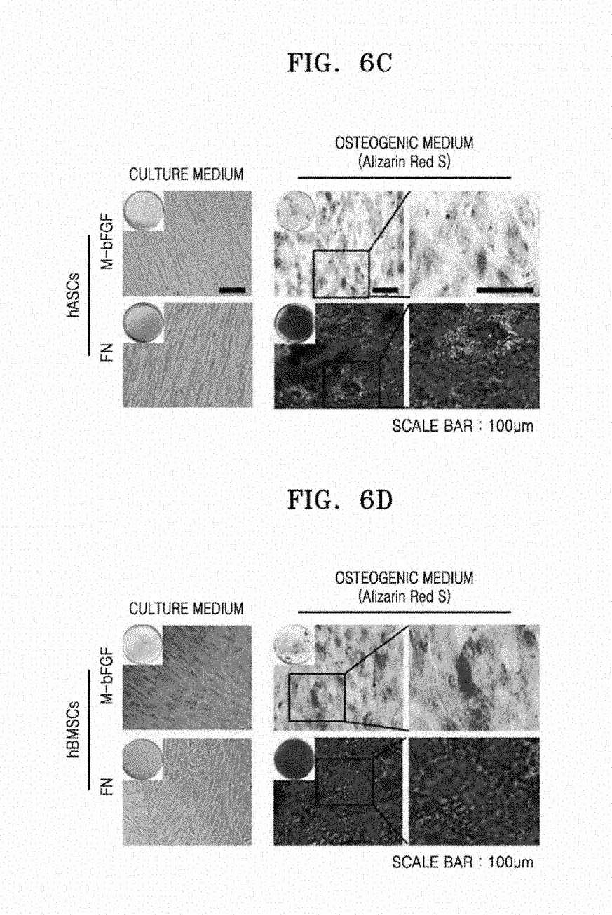

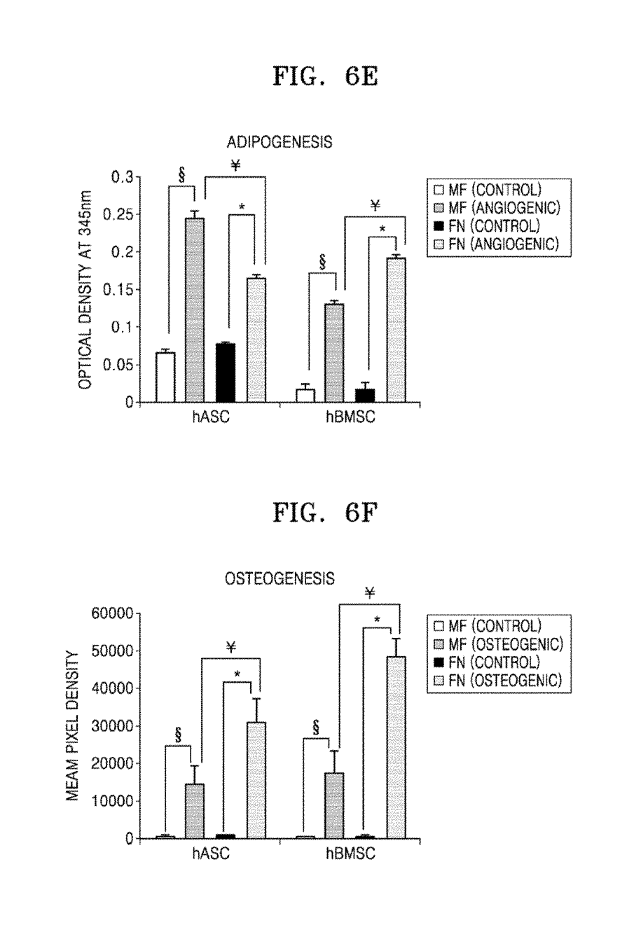

First, the hASCs and the hBMSCs in a culture medium were dispensed on M-bFGF-coated and FN-coated cover slips at a concentration of 1.0×10⁴ cells/cm² and cultured under conditions of 37° C. and 5% CO₂ for 2 days. On the 3rd day of culturing, the culture medium was replaced with an adipose cell differentiation medium (including 10 μg/ml of insulin, 115 μg/ml of methylisobutyl xanthine, 1 M of dexamethasone, and 20 μM of indomethacin) or a bone marrow cell differentiation medium (including 10 nM of dexamethasone, 25 g/ml of ascorbic acid, and 10 mM of L-glycerophosphate), and culturing was continued. The cells were cultured until the 21st day while replacing a differentiation induction medium everyday under the conditions of 37° C. and 5% CO₂. After differentiation, the cells were stained with Oil Red O (Sigma-Aldrich) for detection of lipid droplets or Alizarin Red S (Sigma-Aldrich) for calcium detection. Then, adipose cell differentiation potential and bone marrow cell differentiation potential were quantitatively analyzed. For the quantitative evaluation of adipose cell differentiation potential, the cells were washed with PBS three times, and 500 ml of 100% isopropyl alcohol was added to each well. Thereafter, an OD value of each well was measured at 490 nm by using a NanoDrop ND-1000 spectrophotometer (Thermo Fisher Scientific). For the quantitative evaluation of the bone marrow cell differentiation potential, an image of the stained cells was obtained by using an optical microscope (Nikon Te 2000-U, Japan), and an analysis for calculating a percentage of the stained cells was performed on the obtained image by using the image J software. The results are shown in FIGS. 6A-6F.

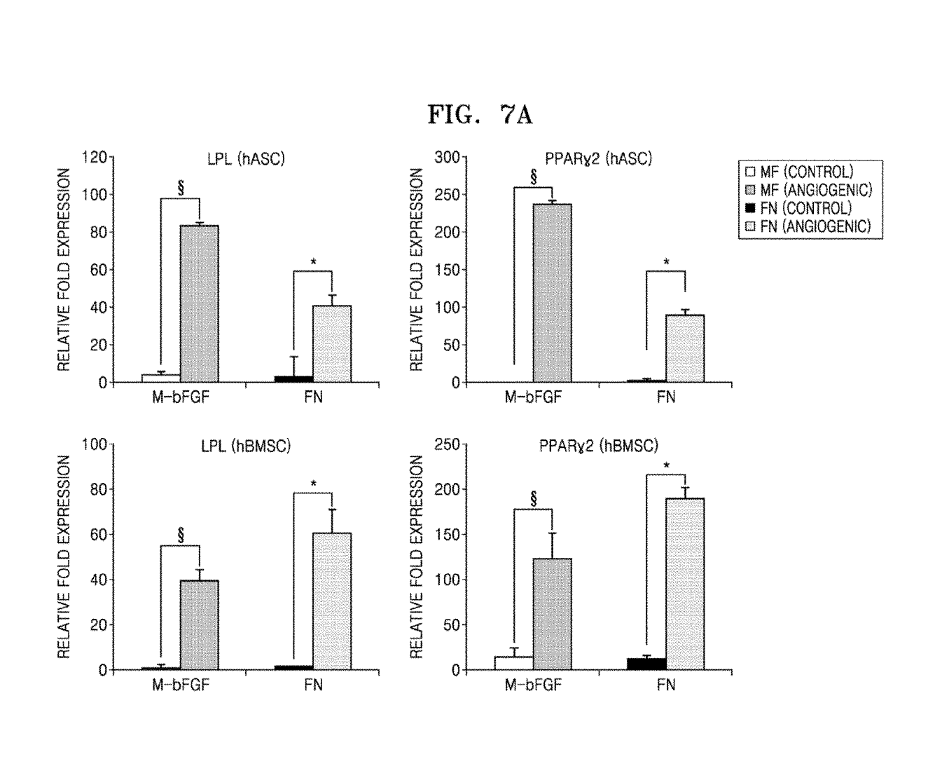

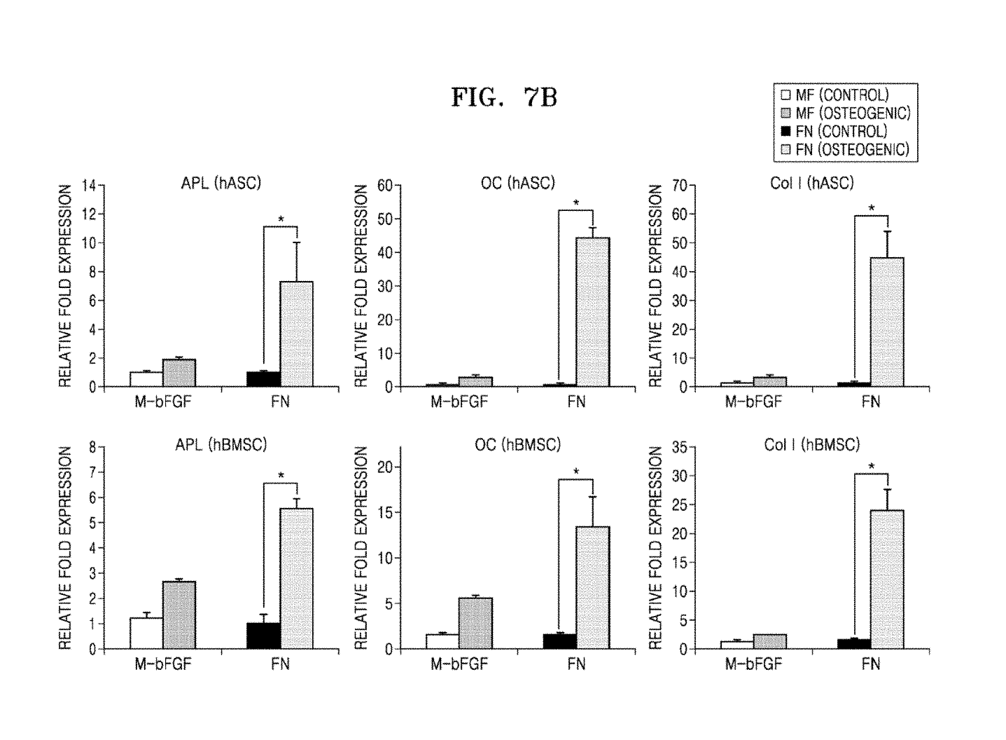

For additional confirmation of the adipose differentiation potential and marrow cell differentiation potential, a quantified reverse transcription-polymerase chain reaction (qRT-PCR) was performed. As control groups, hASCs and hBMSCs cultured in a culture medium for 21 days were used. First, a trizol RNA isolation agent (Invitrogen) was used to extract the total mRNA from the differentiated cells, according to instructions of the manufacturer. A concentration of the isolated RNA was determined at 260 nm by using a NanoDrop ND-1000 spectrophotometer. Then, 1 μg of template RNA was added to a tube of Maxime RT PreMix kit (25081; Intron) to a total volume of 20 μl. Next, cDNA synthesis and RTase inactivation were respectively performed at 45° C. for 60 minutes and at 95° C. for 5 minutes. Target genes and their primers for qRT-PCR are shown in Table 1, and GAPDH was used as a control group. The qRT-PCR was performed by using a iQ™ SYBR Green Supermix kit (Bio-Rad) and a MyiQ™ single color Real-Time PCR Detection System (Bio-Rad) according to instructions of the manufacturer, and the results are shown in FIG. 7.

TABLE 1

| Target gene | SEQ ID NO. |
|---|---|
| Lipoprotein lipase (LPL) forward primer | SEQ ID NO.: 1 |
| Lipoprotein lipase (LPL) reverse primer | SEQ ID NO.: 2 |
| Peroxisome proliferator-activated receptor | SEQ ID NO.: 3 |
| [symbol] 2(PPAR [symbol] 2) forward primer | |
| Peroxisome proliferator-activated receptor | SEQ ID NO.: 4 |
| [symbol] 2(PPAR [symbol] 2) reverse primer | |
| Alkaline phosphatase (ALP) forward primer | SEQ ID NO.: 5 |
| Alkaline phosphatase (ALP) reverse primer | SEQ ID NO.: 6 |
| Osteocalin (OC) forward primer | SEQ ID NO.: 7 |
| Osteocalin (OC) reverse primer | SEQ ID NO.: 8 |
| Collagen type I (Col I) forward primer | SEQ ID NO.: 9 |
| Collagen type I (Col I) reverse primer | SEQ ID NO.: 10 |
| GAPDH forward primer | SEQ ID NO.: 11 |
| GAPDH reverse primer | SEQ ID NO.: 12 |

FIGS. 6A to 6F illustrate the results of analyzing differentiation potentials of stem cells according to cell adhesion control by cell staining, according to an embodiment.

FIGS. 7A and 7B are graphs illustrating the results of measuring differentiation potentials of stem cells according to cell adhesion control by performing qRT-PCR, according to an embodiment.

As shown in FIG. 6A, positive adipose stem cells were observed with strong red staining on the surface of M-bFGF as compared to the surface of FN. In contrast, as shown in FIG. 6B, bone marrow stem cells were observed with strong staining on the surface of FN as compared to the surface of M-bFGF. Also, as shown in FIGS. 6C and 6D, the adipose stem cells and bone marrow stem cells cultured on the surface of FN were stained well with alizarin red S indicating calcium deposition, whereas only a small number of stem cells cultured on the surface of M-bFGF were positive. FIGS. 6E and 6F are graphs that show quantitative analysis of the results, and the adipose stem cells cultured on the surface of M-bFGF showed statistically significant positive staining for lipid vacuole by oil red S analysis as compared to the adipose stem cells cultured on the surface of FN. On the other hand, in the case of bone marrow stem cells, the opposite result was obtained. In addition, both adipose stem cells and bone marrow stem cells cultured on the surface of FN showed statistically significant positive staining for calcium deposition as compared to cells cultured on the surface of M-bFGF.

The results of performing the qRT-PCR to verify the above results are shown in FIGS. 7A-7B. As shown in FIG. 7A, the adipose stem cells cultured on the surface of M-bFGF promoted expression of adipogenic genes LPL and PPAR γ 2 as compared with the adipose stem cells cultured on the surface of FN, and the opposite result was obtained in the case of bone marrow stem cells. In addition, as shown in FIG. 7B, observed expression of osteoblastic genes ALP, OC, and collagen I was significant in both adipose stem cells and bone marrow stem cells cultured on the surface of FN.

As a result of the above description, it may be known that endogenous differentiation potential of stem cells may be controlled according to cell adhesion and may vary depending on a type of the stem cells. Also, as a result of the above description, it may be known that adipose stem cells have competitive adipose cell differentiation potential and bone marrow differentiation potential depending on integrin-mediated adhesion control, whereas differentiation of bone marrow stem cells is up-controlled by integrin signal activation, and thus adipose cell differentiation potential and bone marrow differentiation potential may not be competitive in the bone marrow stem cells.

As described above, according to one or more embodiments, a culture container and a method of classifying mesenchymal stem cells may be effectively used to classify, identify, or specify mesenchymal stem cells by controlling cell adhesion to a culture container when culturing the mesenchymal stem cells.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A method of classifying mesenchymal stem cells by controlling cell adhesion, the method comprising:
culturing mesenchymal stem cells in a culture container coated with a protein for controlling cell adhesion;
analyzing characteristics of the mesenchymal stem cells thus cultured; and
classifying the mesenchymal stem cells according to results of the analysis.

2. The method of claim 1, wherein the protein for controlling cell adhesion is a protein for limiting integrin-mediated cell adhesion or for integrin-mediated cell adhesion.

3. The method of claim 2, wherein the protein for limiting integrin-mediated cell adhesion is a protein in which a polypeptide linker and a growth factor are fused.

4. The method of claim 3, wherein the polypeptide linker is maltose-binding protein (MBP), hydrophobin, or hydrophobic cell penetrating peptides (CPPs).

5. The method of claim 3, wherein the growth factor is a vascular endothelial growth factor (VEGF), a fibroblast growth factor (FGF), an epidermal growth factor (EGF), a platelet-derived endothelial growth factor (PDGF), a hepatocyte growth factor (HGF), an insulin-like growth factor (IGF), or a heparin binding domain (HBD).

6. The method of claim 1, wherein the protein for integrin-mediated cell adhesion is an extracellular matrix protein.

7. The method of claim 6, wherein the extracellular matrix protein is collagen, fibronectin, or laminin.

8. The method of claim 1, wherein the analyzing of the characteristics of the mesenchymal stem cells is analyzing of a cytoskeletal structure, cell morphology, intracellular signal transduction, or cell differentiation potential.

9. The method of claim 1, wherein the classifying of the mesenchymal stem cells comprises classifying mesenchymal stem cells having particular morphology or differentiation potential as cultured by integrin-mediated cell adhesion; or mesenchymal stem cells having particular morphology or differentiation potential as cultured while limiting integrin-mediated cell adhesion.

10. The method of claim 1, wherein the mesenchymal stem cells are adipose stem cells, bone marrow stem cells, neural stem cells, placental stem cells, or cord blood stem cells.
